(19) United States
(12) Patent Application Publication
(10) Pub. No.: US 2021/0137773 A1
(43) Pub. Date: May 13, 2021

(54) MASSAGE DEVICE

(71) Applicant: FITSON SINGAPORE PTE. LTD., Singapore (SG)

(72) Inventor: Yi Wen CHAN, Singapore (SG)

(73) Assignee: FITSON SINGAPORE PTE. LTD., Singapore (SG)

(21) Appl. No.: 17/050,964

(22) PCT Filed: Dec. 28, 2018
(86) PCT No.: PCT/SG2018/050639

§ 371 Date: Oct. 27, 2020

Related U.S. Application Data
(60) Provisional application No. 62/666,637, filed on May 3, 2018.

(51) Int. Cl. A61H 7/00; A61M 1/06

(52) U.S. Cl. CPC A61H 7/008; A61M 1/064; A61H 2205/062; A61H 2201/1207; A61H 2201/0207; A61H 2205/082

(57) ABSTRACT

A massage device is disclosed that is made by converting an existing breast pump using a massage adapter ring that allows for the easy conversion to and from a massage device to breast pump.

### CROSS-REFERENCE TO RELATED APPLICATIONS

This application is a 35 U.S.C. 371 National Phase Entry Application from PCT/SG2018/050639, filed Dec. 28, 2018, which designates the United States and claims the benefit of U.S. Provisional Application No. 62/666,637 filed on May 3, 2018, the disclosures of which are incorporated herein in their entirety by reference.

### TECHNICAL FIELD

This invention relates to a massage device, in particular to a massage device that uses negative vacuum to knead and apply pressure to a part of the body. The massage device can be converted to and from a breast pump that assists mothers in expressing milk.

### BACKGROUND

Various types of massage devices are available to consumers in the market. These can range from large massage chairs and tables to small handheld devices but share the common characteristic of being designed for a single purpose.

One way of providing a massage function is by making a change in air pressure within a confined space, and this can be a positive vacuum where a motor pushes air out; or this can be negative vacuum or a suctioning motion, where the air is used to create suction effect to knead and apply pressure.

Modern breast pump devices typically use a negative vacuum to draw milk into a bottle and assist a mother in expressing breast milk for a baby. It would be ideal if a breast pump device can be converted into a massage device, although it is noted that the design of current conventional breast pump does not allow for easy conversion.

Hence, there is a need for massage device that can be converted to and from an existing breast pump, without undue effort from the user.

### BRIEF SUMMARY

The above and other problems are solved and an improvement in the art is made by a massage device in accordance with this invention. A first advantage of the massage device in accordance with this invention is that a breast pump can be converted into a massage device. This allows dual use of an existing product, and lets the mother massage tender areas before or after expressing milk for her child. A second advantage of the massage device in accordance with this invention is that the massage adapter ring provides a tight seal for the diaphragm to ensure sufficient negative vacuum is created in order to provide a massage function.

The present massage device is based on converting a breast pump device or some pump capable of providing a change in air pressure, where a massage adapter ring can be fitted onto a diaphragm and converting the diaphragm into a massage device. The vacuum can be applied in increasing or varying amounts to provide steadily increase or decrease or rhythmic massage function.

In accordance with an embodiment of the present invention, there is provided a massage device for use with a breast pump comprising an enclosed chamber formed by a cap and a flexible diaphragm, an adapter ring adapted to hold the diaphragm against the cap to form the enclosed chamber, an airway from the enclosed chamber in communication with the breast pump, where the enclosed chamber expands and contracts to perform a massage function. Preferably, the enclosed chamber expands and contracts in accordance with the operation of the breast pump. In an alternate embodiment, the flexible diaphragm has a rim to allow the adapter ring to hold the diaphragm against the cap. In yet further embodiments, the cap has a tab and the adapter ring has a corresponding catch to accept tab. In other embodiments, the corresponding catch is L-shaped such that the adapter ring is rotated relative to the cap to secure the adapter ring to the cap. In further alternative embodiments, the cap has a plurality of tabs and the adapter ring has a plurality of catches that correspond to each tab.

In a further aspect, the flexible diaphragm has an indentation to better allow contact with a target massage area. Alternatively, an embodiment of the invention describes the flexible diaphragm that has a groove for improved contact with a target area for massage. In yet another aspect, the flexible diaphragm has a raised ridge for improved contact with a target area for massage. A further aspect of an embodiment of the invention the flexible diaphragm has an inner raised ridge and an outer raised ridge, wherein the amount of vacuum by the breast pump would determine which ridge would flex.

In other embodiments in accordance with the invention, the breast pump operates in a rhythm to provide a massage. In yet further embodiments, the breast pump operates to provide a prolonged constant vacuum to provide a massage. Alternatively, the breast pump gradually changes the vacuum to provide a massage. In another embodiment, the cap and diaphragm is used with an external attachment to express milk

### BRIEF DESCRIPTION OF THE DRAWINGS

The invention is illustrated by way of example with reference to the accompanying drawings which show a massage device constructed in accordance with the invention. The accompanying drawings, which are incorporated herein and form part of the specification, illustrate various embodiments of the present disclosure and, together with the description, further serve to explain the principles of the disclosure and to enable a person skilled in the pertinent art to make and use the embodiments disclosed herein. In the drawings, like reference numbers indicate identical or functionally similar elements.

FIG. 1 is the overview system of a breast pump converted into a massage device with the massage adapter ring fitted;

FIGS. 2A and 2B is the profile of the massage adapter ring of the massage device;

FIG. 3 is a cross section of the massage adapter ring and silicone diaphragm.

FIGS. 4A, 4B, 4C, and 4D is a cross section of the massage adapter ring and silicone diaphragm in various stages of use.

FIG. 5 is a side view of a breast pump for use with an embodiment of the invention.

FIGS. 6A, 6B, 6C, and 6D is a step by step view of how an embodiment of the invention is used.

### DETAILED DESCRIPTION

The various configurations discussed in these non-limiting examples can be varied and are used to illustrate at least one embodiment and are not intended to limit the scope thereof.

Referring to the drawings, FIG. 1 shows a simplified view of a typical breast pump system 100 with the massage adapter ring 7 attached. In the drawing, it can be seen that an electric breast pump 1 with control buttons shown for controlling the operations of the electric breast pump 1. The electric breast pump 1 is able to provide a vacuum or a change in pressure through a valve port or valve pump (shown in FIG. 5) which may cause a negative vacuum along a flexible silicone tubing 2 that is connected to the electric breast pump 1 one end and the other end ending in a funnel cap or cap 3. The control buttons also allow the electric breast pump 1 to provide the negative vacuum in a variety of ways, including a steady rhythmic manner with constant pressure or vacuum; an increasing pressure or vacuum; and intermittent pressure or vacuum, or a decreasing pressure or vacuum, or any other customized timing chosen or selected by the user. A tubing valve may be positioned at the back of the funnel cap 3 for directing the flow of air or for assisting in the change in air pressure. The funnel cap 3 has a silicone diaphragm 4, and up to this stage, all the parts shown may belong or may be used in a breast pump system. Also shown installed is a massage ring adapter 7 that converts the system into a massage device. The massage adapter ring 7 can provide the proper seal that will allow the negative vacuum to operate the undulation of the silicone diaphragm. Further details on this are described later using FIGS. 4A-4D. The massage can limit the amount of skin that is in contact with the silicone diaphragm to allow for a proper seal. Further examples of the massage adapter ring 7 can be seen in FIGS. 2A and 2B. Also shown are some exemplary dimensions, as well as the profile of the massage adapter ring 7. The cap securing catch 8 on the massage adapter ring 7 provides the proper tension seal to the silicone diaphragm when the massage adapter ring 7 is fitted to the funnel cap 3. The funnel cap 3 can be secured or mated to through a variety of means, including tabs, snap fasteners, screw threads, clip etc, and the massage adapter ring 7 is able to fit securely and tightly onto the funnel cap 3. In this instance, a single silicone tube and corresponding funnel cap and diaphragm is described, although the electric breast pump can be connected to more than one silicone tube the same time, for example having two silicone tubes connected to the electric breast pump, each silicone tube with its own funnel cap and diaphragm arrangement. In these instances, either one or both diaphragms can be converted to a massage function.

The funnel cap 3 can have tabs, and the massage adapter ring 7 is able to fit securely and tightly onto the funnel cap 3 by having the tabs slide or secure onto the catch 8. The catch 8 can be an L-shaped groove that allows the tab to slide in and lock the adapter ring in place. Multiple tabs and catches can also be used as seen in FIGS. 2A and 2B where three catches are shown on the adapter ring. The massage adapter ring 7 is also shown with a lift tab as it can allow a better grip for attaching and removing of the massage adapter ring from the funnel cap 3 or the silicone diaphragm 4.

Taking a closer look, FIG. 3 shows a cross section of the massage adapter ring 7 with the silicone diaphragm 4. Also shown is the bottom edge 5 of the massage adapter ring 7 that traps the rim of the diaphragm 4 against the cap 3. In some embodiments, the diaphragm and funnel cap can be made from a single piece, this would provide better sealing and prevent any leakage. In this embodiment, the massage adapter ring may still be used to provide the grooves and ridges, and to vary its height to allow appropriate contact between the diaphragm and the target area for massage.

The massage ring adapter can secure the silicone diaphragm to the funnel cap and ensure a vacuum seal. The massage adapter ring can maintain a tight seal closure with the tab catch on the funnel cap providing an enclosed space formed by the diaphragm 4 and the funnel cap 3. The massage adapter ring can provide the right amount of undulating force provided by the silicone diaphragm through the height of the ring. The massage adapter ring can limit the amount of skin contact to the silicone diaphragm by varying the height of the ring relative to the diaphragm.

In some embodiments, a heating element can be added such that the massage adapter ring or the silicone diaphragm would be heated or warmed for added comfort to the user. This heating element can be connected to the same power source as the breast pump or even from the breast pump itself.

FIGS. 4A, 4B, 40, and 4D show a cross section of the silicone diaphragm 4 working with the massage adapter ring 7 to provide a massage function. When skin 9 is brought into contact with the silicone diaphragm (FIG. 4B), the exposed ridges of the diaphragm can aid in the massage function by providing a secondary seal. The profile of the massage adapter ring can prevent the surface of the skin from pressing too near or too far from or into the diaphragm using the edge of the massage adapter ring or the massage adapter ring lip 6. The ridges 10 of the diaphragm can seal the skin to create a vacuum. When a low vacuum is exerted by the electric breast pump, the inner ridge of the diaphragm starts to pull the skin and create a suction effect 11 also known as negative vacuum. With a higher negative vacuum, the inner ridge 12 of the diaphragm pulls away from the skin while the outer ridge of the diaphragm 13 pulls the skin towards the center of the diaphragm creating a higher vacuum pull.

The various access ports for the electric breast pump 1 can be seen in a side view shown in FIG. 5. A valve port 14 can be seen which assists the electric breast pump 1 in providing a change in pressure along the silicone tube (not shown in FIG. 5) by only allowing the flow of air in a particular direction. In the case of an electric breast pump, this flow of air causes a negative vacuum along the silicone tube which in turn causes the diaphragm to contract accordingly and assist the user in expressing milk. This same negative vacuum can be used when the massage adapter ring (not shown in FIG. 5) is attached to the diaphragm, converting the system into a massage device. A electrical or electronics port 15 can be seen, which allows the electrical breast pump to be powered by sending electrical current through the port, or allows the electrical breast pump to be programed using an external computing device.

The conversion of the cap 3 and diaphragm 4 used in a breast pump into a massage device can be seen in FIGS. 6A, 6B, 60, and 6D. The diaphragm 4 is fitted onto the funnel cap or cap 3 as usual for usage as a breast pump in FIG. 6A. However, instead of fitting the cap onto a breast pump funnel (not shown), a massage adapter ring 7 is installed instead. This is done by fitting the massage adapter ring 7 over and around the edge of the diaphragm 4, such that the tabs of the cap 3 engage the catch 8 of the massage adapter ring 7 and twisting the massage ring 7 relative to the cap 3, such that the tabs of the cap 3 slide along the catch 8 and secures the massage adapter ring 7 in place, thereby holding the diaphragm 4 in place and forming the enclosed space with the cap 3 as seen in FIG. 6B. The twisting motion may be aided by the lifting tab on the massage adapter ring 7, and a click can be felt or heard when the massage adapter ring 7 is in secured in place. The flexible tubing 2 that is used for the breast pump is then attached to the funnel cap 3, as per when it is used as a breast pump in FIG. 6C. The cap 3 (with the flexible tubing 2 attached) is pressed against the target area for massage, with the diaphragm 4 pressing against the target area, i.e. the skin of the person, and the breast pump is activated. This would provide a gentle cupping and/or kneading sensation, depending on the type of diaphragm used as well as the operation mode of the breast pump. The breast pump can also have a specific massage mode that is activated when the massage adapter ring 7 is fitted onto the cap 3.

The described apparatus and methods are used for a variety of massage functions, particularly those which use negative vacuum. Other embodiments can also use positive air pressure in order to effect a massage function, especially with a pulsing motion. This is dependent on the operation of the breast pump, whether it draws in air or it pumps out air. Other embodiments include adapting the breast pump to draw in water and pump out water. This is useful if the water is warmed up in the process to provide a warm massage cupping or kneading function.

Thus, the present invention has been fully described with reference to the drawing figures. Although the invention has been described based upon these preferred embodiments, to those of skill in the art, certain modifications, variations, and alternative constructions would be apparent, while remaining within the spirit and scope of the invention.

1. A massage device configured for use with a breast pump comprising:
an enclosed chamber formed by a cap and a flexible diaphragm;
an adapter ring adapted to hold the flexible diaphragm against the cap to form the enclosed chamber;
an airway from the enclosed chamber configured for communication with the breast pump;
wherein said enclosed chamber is configured to expand and contract to perform a massage function.

2. The massage device according to claim 1, wherein said enclosed chamber is configured to expand and contract in accordance with operation of the breast pump when the massage device is in communication with the breast pump.

3. The massage device according to claim 1, wherein the flexible diaphragm has a rim to allow the adapter ring to hold the flexible diaphragm against the cap.

4. The massage device according to claim 1, wherein the cap has a tab and the adapter ring has a corresponding catch to accept the tab.

5. The massage device according to claim 4, wherein the corresponding catch is L-shaped such that the adapter ring is rotated relative to the cap to secure the adapter ring to the cap.

6. The massage device according to claim 4, wherein the cap has a plurality of tabs and the adapter ring has a plurality of catches such that each of the plurality of catches corresponds to each tab of the plurality of tabs.

7. The massage device according to claim 1, wherein the flexible diaphragm has an indentation at a surface of the diaphragm facing away from the cap.

8. The massage device according to claim 1, wherein the flexible diaphragm has a groove at a surface of the diaphragm facing away from the cap.

9. The massage device according to claim 1, wherein the flexible diaphragm has a raised ridge at a surface of the diaphragm facing away from the cap.

10. The massage device according to claim 1, wherein the flexible diaphragm has an inner raised ridge and an outer raised ridge, wherein either the inner raised ridge or the outer raised ridge is configured to flex based on an amount of vacuum provided by the breast pump.

11. The massage device according to claim 2, wherein said massage device is configured to provide a rhythmic massage to a target massage area corresponding to a rhythm of the breast pump in communication with the massage device.

12. The massage device according to claim 2, wherein said massage device is configured to provide a rhythmic massage to a target massage area wherein the rhythmic massage comprises a constant or intermittent pressure or vacuum provided by the breast pump in communication with the massage device.

13. The massage device according to claim 2, wherein said breast pump gradually changes the vacuum to provide a massage.

14. The massage device according to claim 1, wherein the cap and the flexible diaphragm are configured to attach to an external attachment suitable for expressing milk.

15. A method of providing a massage to a target massage area comprising:
providing a massage device comprising:
an enclosed chamber formed by a cap and a flexible diaphragm;
an adapter ring adapted to hold the flexible diaphragm against the cap to form the enclosed chamber; and
an airway from the enclosed chamber;
placing the airway from the enclosed chamber in communication with a breast pump; and
operating the breast pump;
wherein said enclosed chamber is configured to expand and contract during operation of the breast pump to provide a massage to the target massage area.

16. The method of claim 15, wherein the flexible diaphragm has an inner raised ridge and an outer raised ridge, further comprising providing different amounts of vacuum during operation of the breast pump such that either the inner raised ridge or the outer raised ridge flexes based on different amounts of vacuum provided by the breast pump.

17. The method of claim 15, further comprising providing a rhythmic massage to the target massage area corresponding to a rhythm of the breast pump in communication with the massage device during operation of the breast pump.

18. The method of claim 15, further comprising providing a constant vacuum by the breast pump in communication with the massage device during operation of the breast pump.

19. The method of claim 15, further comprising changing vacuum pressure provided by the breast pump to the massage device to provide a change in massage pressure or rhythm corresponding to changes in the changing vacuum pressure provided by the breast pump.

20. The method of claim 15, further comprising attaching an external attachment to the cap and the flexible diaphragm and expressing breast milk using the external attachment during operation of the breast pump.
